(19) United States
(12) Patent Application Publication
(10) Pub. No.: US 2009/0082869 A1
(43) Pub. Date: Mar. 26, 2009

(54) DOUBLE ENDED PYRAMID ADAPTER

(76) Inventors: Tracy C. Slemker, Clayton, OH (US); Christos Lemonis, Jupiter, FL (US); Lucinda Busch, Clayton, OH (US); Paul L. Galloway, Clayton, OH (US); Steven Steinbarger, Wilmington, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 12/210,608

(22) Filed: Sep. 15, 2008

Related U.S. Application Data
(60) Provisional application No. 60/993,799, filed on Sep. 14, 2007.

(51) Int. Cl. A61F 2/44

(52) U.S. Cl. 623/17.16; 623/17.11

(57) ABSTRACT

A prosthetic coupling providing height and rotation adjustability. Exemplary embodiments include a Double Ended Pyramid Adapter (DEPR) allowing a prosthesis to be height adjusted and rotated using a single adapter. An exemplary DEPR also has an anti-rotation locking feature that selectively inhibits rotation of the components with respect to one another, and can selectively be unlocked to allow rotation when desired. An exemplary DEPR adapter allows for height and rotation adjustment, and may be utilized with transfemoral patients for height adjustments between the distal end of the socket and the prosthetic knee.

### CROSS REFERENCE TO RELATED APPLICATION

This application claims the benefit of U.S. Provisional Application No. 60/993,799, filed Sep. 14, 2007, the disclosure of which is hereby incorporated by reference.

### BACKGROUND

### Field of the Invention

The exemplary embodiments disclosed herein are directed to prosthetic components and, more specifically, to prosthetic adapters for receiving opposing pyramids, where the prosthetic adapter provides rotational and vertical adjustability.

### INTRODUCTION TO THE INVENTION

In a first aspect, a prosthetic coupling may include a proximal component including a first pyramid receiver and a first threaded portion; and a distal component including a second pyramid receiver and a second threaded portion, the second threaded portion engaging the first threaded portion and allowing at least one of rotational and vertical adjustability between the first pyramid receiver and the second pyramid receiver.

In a detailed embodiment of the first aspect, the prosthetic coupling may further include a lock operative to selectively inhibit at least one of rotational adjustability of the proximal component relative to the distal component and vertical adjustability between the proximal component and the distal component. In a further detailed embodiment, the lock may include a key mounted to one of the proximal component and the distal component and at least one notch oh the other of the proximal component and the distal component. The notch may receive at least a portion of the key to inhibit at least one of rotational adjustability of the proximal component relative to the distal component and vertical adjustability between the proximal component and the distal component. In still a further detailed embodiment, the key may be movably mounted to the one of the proximal component and the distal component and the key may movable between an engaged position in which the key is at least partially received within the notch to inhibit least one of rotational adjustability of the proximal component relative to the distal component and vertical adjustability between the proximal component and the distal component, and a disengaged position allowing least one of rotational adjustability of the proximal component relative to the distal component and vertical adjustability between the proximal component and the distal component. In yet a further detailed embodiment, The prosthetic coupling may further include a bias operative to urge the key towards the engaged position. In a further detailed embodiment, the key may be pivotable between the engaged position and the disengaged position. In still a further detailed embodiment, the at least one notch may be located on a circumferential surface of the distal component at least partially between the second pyramid adapter and the second threaded portion. In yet a further detailed embodiment, the key may be mounted to the proximal component and the key may extend at least partially beyond the first threaded portion in a direction away from the first pyramid receiver. In another detailed embodiment, the proximal component may include a first index mark and the distal component may include a second index mark and alignment of the first index mark and the second index mark may indicate that the key is aligned with the notch.

In another detailed embodiment of the first aspect, the first threaded portion or the second threaded portion may include an internally threaded, generally cylindrical cavity and the other may include ah externally threaded projection. In a further detailed embodiment, the cylindrical cavity may include an internally threaded portion and a smooth portion. In yet a further detailed embodiment, the smooth portion may have a diameter greater than a diameter of the internally threaded portion. In another detailed embodiment, the projection may include an externally threaded portion and a smooth portion. In a further detailed embodiment, the smooth portion may have a diameter greater than a diameter of the externally threaded portion.

In another detailed embodiment of the first aspect, the proximal component may include a first index mark and the distal component may include a second index mark. In another detailed embodiment of the first aspect, the first pyramid receiver may include a hollowed space at least partially defined by an interior circumferential wall, the interior circumferential wall including a first ring having an axially arcuate shape with an axially decreasing diameter towards a second arcuate ring, a step change in axial height interposing the first arcuate ring and the second arcuate ring. In another detailed embodiment of the first aspect, the first pyramid adapter may include at least one threaded hole adapted to receive a set screw.

In another, detailed embodiment of the first aspect, the proximal component may include a clamp, the clamp including a generally cylindrical axial opening including an interior surface that is at least partially threaded. In a further detailed embodiment, the generally cylindrical axial opening may be partially threaded and partially smooth. In another further detailed embodiment, the clamp may include a generally axial groove extending from an exterior of the clamp to the generally cylindrical axial opening. In a still further detailed embodiment, the clamp may include a through hole extending perpendicular to the groove. In another detailed embodiment, the clamp may be integrally formed with the first pyramid adapter.

In a second aspect, a prosthetic coupling may include a first component including a first pyramid adapter and an internal cavity having an opening; a second component including a second pyramid adapter and a projection at least partially received within the internal cavity; and a key mounted to one of the first component and the second component to selectively inhibit rotation between the first component and the second component.

In a detailed embodiment of the second aspect, the prosthetic coupling may further include a notch on the other of the first component and the second component. The notch may be adapted to releasably receive at least a portion of the key. In another detailed embodiment of the second aspect, the prosthetic coupling may further include a through hole in the other of the first component and the second component and the through hole is adapted to releasably receive at least a portion of the key.

In another detailed embodiment of the second aspect, the key may be pivotably mounted to the one of the first component and the second component. In a further detailed embodiment, the key may be mounted to the first component and the second component may include the at least one notch. In a still further detailed embodiment, at least one of the first pyramid adapter and the second pyramid adapter may be a pyramid receiver. In a yet further detailed embodiment, the pyramid receiver may include a hollowed space at least partially defined by an interior circumferential wall, the interior circumferential wall including a first ring having an axially arcuate shape with an axially decreasing diameter towards a second arcuate ring, a step change in axial height interposing the first arcuate ring and the second arcuate ring. In a further detailed embodiment, the pyramid receiver may include at least one threaded hole adapted to receive a set screw.

In another detailed embodiment of the second aspect, the first component may include a first alignment mark and the second component may include a second alignment mark and alignment of the first alignment mark and the second alignment mark may indicate that the key is aligned with the notch. In a: further detailed embodiment, the first pyramid adapter may include at least one threaded hole and the first alignment mark may be substantially aligned with the at least one threaded hole.

In another detailed embodiment of the second aspect, the internal cavity may include an internally threaded portion. In a further detailed embodiment, the internal cavity may include an internal smooth portion. In a still further detailed embodiment, the internal smooth portion may have a diameter greater than a diameter of the internally threaded portion.

In another detailed embodiment of the second aspect, the projection may include an externally threaded portion. In a further detailed embodiment, the projection may include an external smooth portion. In a still further detailed embodiment, the external smooth portion may interpose the externally threaded portion and the second pyramid adapter. In a further detailed embodiment, the external smooth portion may have a diameter greater than a diameter of the externally threaded portion.

In a third aspect, a prosthetic coupling adapter may include a first component including a first pyramid receiver including a hollow axial opening and a plurality of generally radially oriented, threaded holes extending to the hollow axial opening, and a clamp mounted axially adjacent to the first pyramid receiver including an axial cylindrical opening including an internally threaded portion and an internal smooth portion, an axial groove extending from an exterior of the clamp to the axial cylindrical opening, and a key pivotably mounted within the groove; and a second component including a second pyramid receiver including a hollow axial opening and a plurality of generally radially oriented threaded holes extending to the hollow axial opening, and a projection mounted axially adjacent the second pyramid receiver including an externally threaded portion engaging the internally threaded portion, and an external smooth portion including at least one notch, the notch being selectively engageable with at least a portion of the key.

In a detailed embodiment of the third aspect, the internal smooth portion may have a diameter greater than a diameter of the internally threaded portion. In another detailed embodiment of the third aspect, the external smooth portion may have a diameter greater than a diameter of the externally threaded portion. In another detailed embodiment of the third aspect, the first pyramid receiver may include a hollowed space at least partially defined by an interior circumferential wall, the interior circumferential wall including a first ring having an axially arcuate shape with an axially decreasing diameter towards a second arcuate ring, a step change in axial height interposing the first arcuate ring and the second arcuate ring.

In a fourth aspect, a method of adjusting a prosthetic coupling may include operating a lock to a disengaged position to permit rotation of a first threaded portion of a first component relative to a corresponding second threaded portion of a second component, the lock having an engaged position in which the lock inhibits relative rotation of the first component relative to the second component, the first component including a first pyramid receiver and the second component including a second pyramid receiver; and rotating the first component relative to the second component, thereby engaging the first threaded portion and the second threaded portion.

In a detailed embodiment of the fourth aspect, the method may further include, prior to the step of operating the lock, aligning the first threaded portion with the second threaded portion. In a further detailed embodiment, the method may further include operating the lock to the engaged position. In a still further detailed embodiment, the method may further include, prior to the step of operating the lock to the engaged position, aligning a key mounted to one of the first component and the second component with a notch on the other of the first component and the second component. In a still further detailed embodiment, the other of the first component and the second component may include a plurality of notches and the step of aligning the key includes selecting one of the plurality of notches.

In another detailed embodiment of the fourth aspect, the lock may include a key pivotably mounted to one of the first component and the second component and a notch on the other, of the first component and the second component, the notch may be adapted to receive a portion of the key, and the step of operating the lock to a disengaged position may include pivoting the key.

### BRIEF DESCRIPTION OF THE DRAWINGS

The detailed description refers to the Figures in which:

FIG. 1 is a frontal view of a first exemplary prosthetic adapter in accordance with the instant invention;

FIG. 2 is a left profile view of the first exemplary prosthetic adapter of FIG. 1;

FIG. 3 is an elevated perspective view from the right side of the first exemplary prosthetic adapter of FIG. 1;

FIG. 4 is an bottom perspective view from the left side of the first exemplary prosthetic adapter of FIG. 1;

FIG. 5 is a left rear profile view of a proximal component of the exemplary prosthetic adapter of FIG. 1;

FIG. 6 is a right rear profile view of a proximal component of the exemplary prosthetic adapter of FIG. 1;

FIG. 7 is a left profile view of the proximal, component of the exemplary prosthetic adapter of FIG. 6;

FIG. 8 is a frontal view of the proximal component of the exemplary prosthetic adapter of FIG. 6 (without the key);

FIG. 9 is a bottom view of the proximal component of the exemplary prosthetic adapter of FIG. 6 (without the key);

FIG. 10 is a vertical cross-sectional view of the proximal component of the exemplary prosthetic adapter of FIG. 6;

FIG. 11 is an additional vertical cross-sectional view of the proximal component of the exemplary prosthetic adapter of FIG. 6;

FIG. 12 is a horizontal cross-sectional view of the proximal component of the exemplary prosthetic adapter of FIG. 6;

FIG. 13 is a frontal view of a distal component of the exemplary prosthetic adapter of FIG. 1;

FIG. 14 is a bottom view of the distal component of the exemplary prosthetic adapter of FIG. 13;

FIG. 15 is a blown-up frontal view of a segment of the distal component of the exemplary prosthetic adapter of FIG. 13;

FIG. 16 is a bottom perspective view of the distal component of the exemplary prosthetic adapter of FIG. 13; and

FIG. 17 is a vertical cross-sectional view of the distal component of the exemplary prosthetic adapter of FIG. 13.

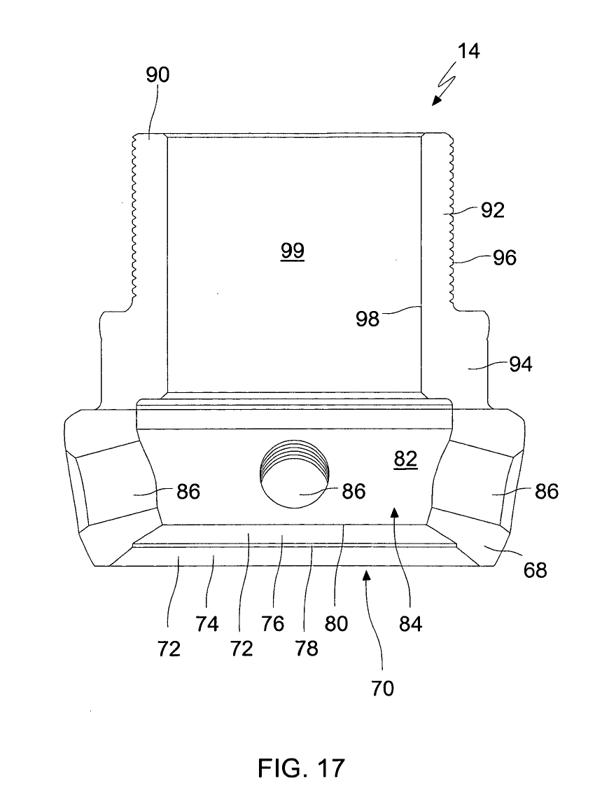

### DETAILED DESCRIPTION

The exemplary embodiments are described and illustrated below to encompass prosthetic adapters as coupling components for prosthetic limb assemblies, and methods for using prosthetic adapters. Of course, it will be apparent to those, of ordinary skill in the art that the embodiment discussed below is exemplary in nature and may be reconfigured without departing from the scope and spirit of the invention. However, for clarity and precision, the exemplary embodiments as discussed below may include optional steps, methods, and features that one of ordinary skill should recognize as not being a requisite to fall within the scope of the invention.

Exemplary embodiments include a Double Ended Pyramid Adapter (DEPR) allowing a prosthesis to be height adjusted and rotated using a single adapter. An exemplary DEPR also has an anti-rotation locking feature that selectively inhibits rotation of the components with respect to one another, and can selectively be unlocked to allow rotation by a prosthetic fitter. With higher profile prosthetic feet, length may become an issue with longer transtibial patients. An exemplary DEPR adapter allows for height and rotational adjustment, and may be utilized with transfemoral patients for height adjustments between the distal end of the socket and the prosthetic knee.

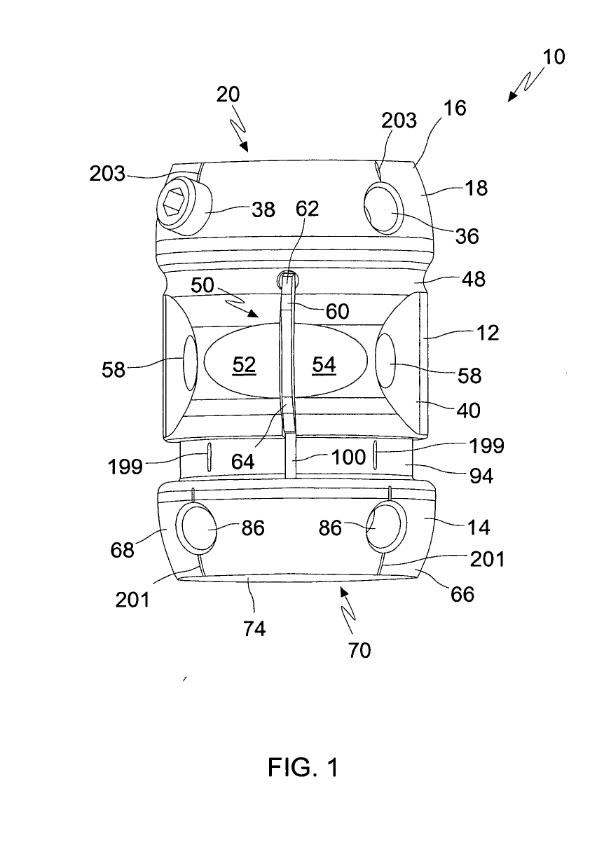

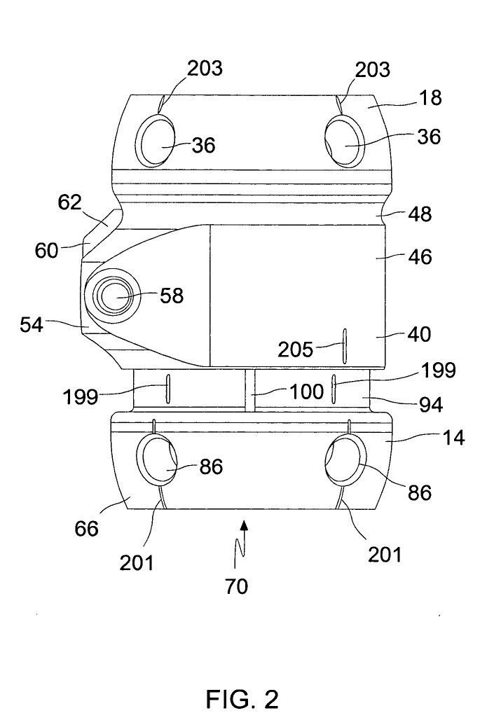

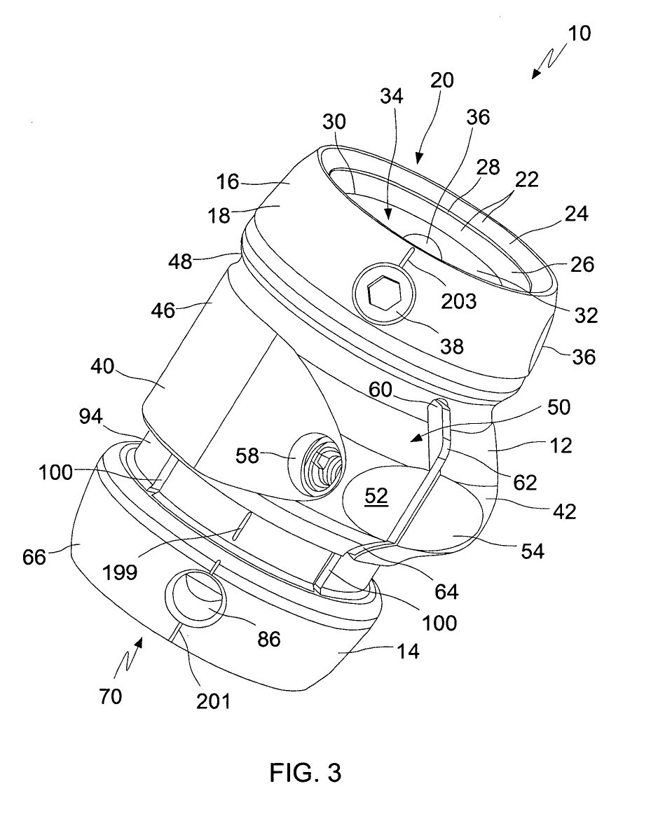

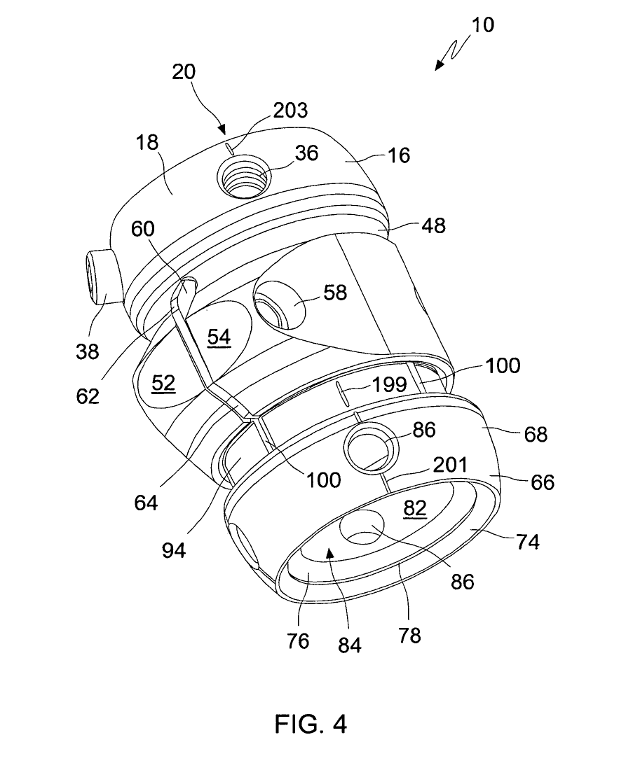

Referencing FIGS. 1-4, an exemplary prosthetic adapter 10 includes a proximal component 12 and a distal component 14 that are mounted to one another by way of a threaded attachment and compression lock. As will be discussed in more detail hereafter, the exemplary adapter 10 provides novel functionality in that both rotational adjustment and height adjustment may be achieved and thereafter fixed in relative orientation to provide a stable platform to which further prosthetic components are mounted, namely an opposing pair of prosthetic pyramids.

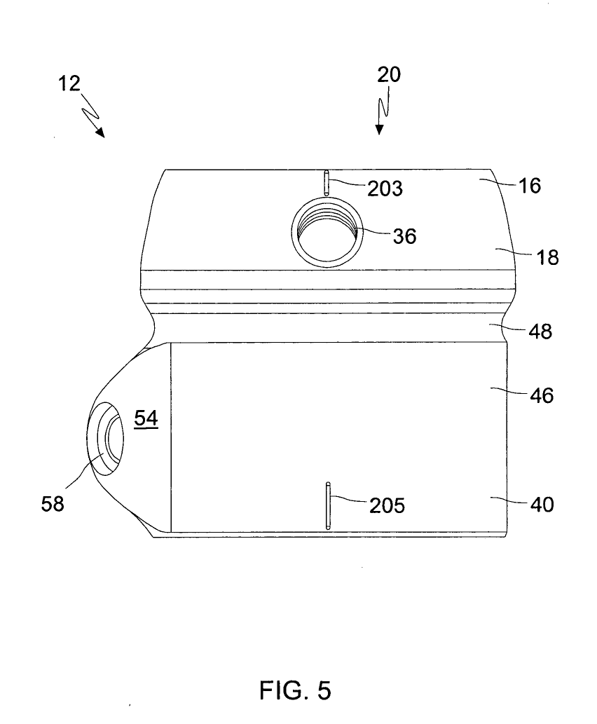

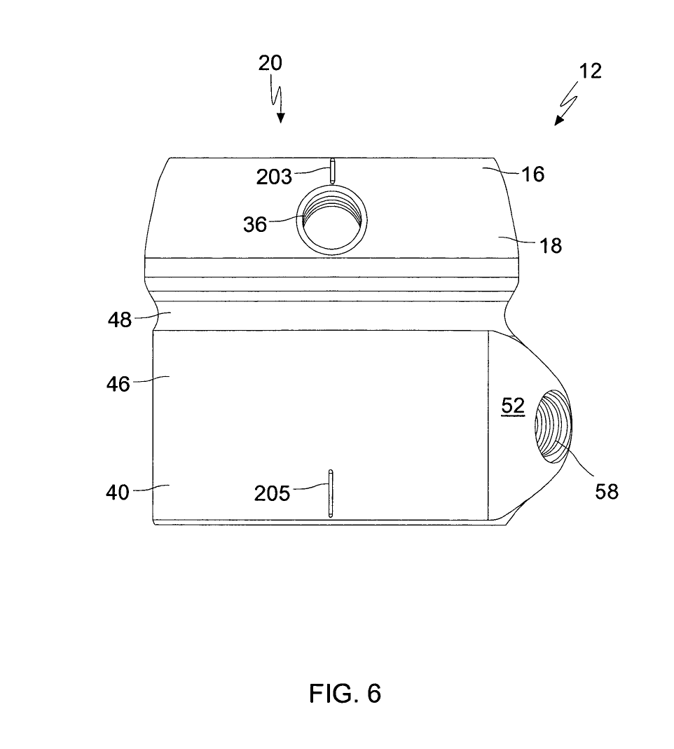

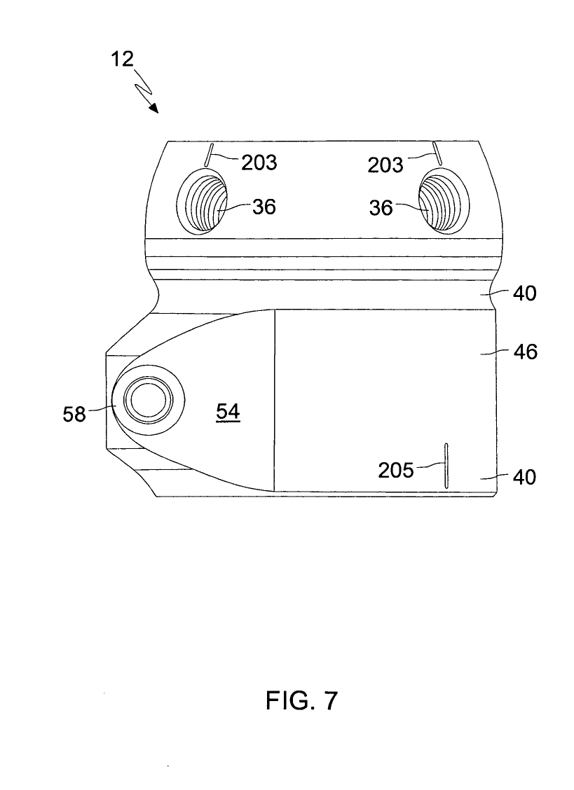

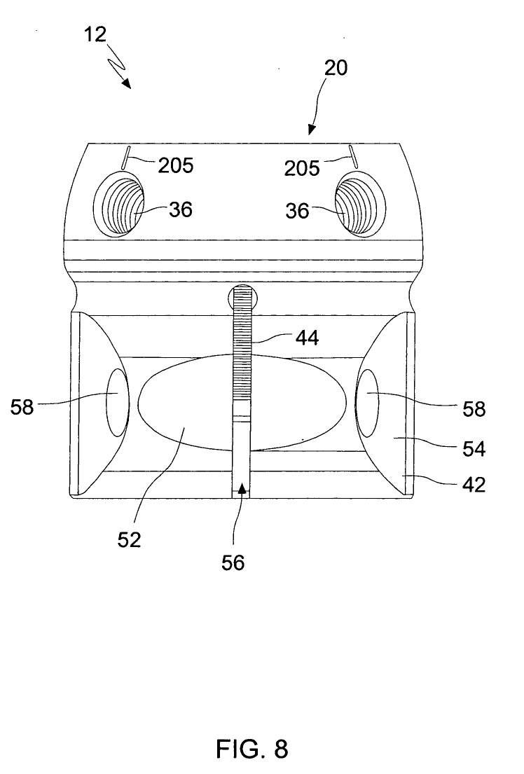

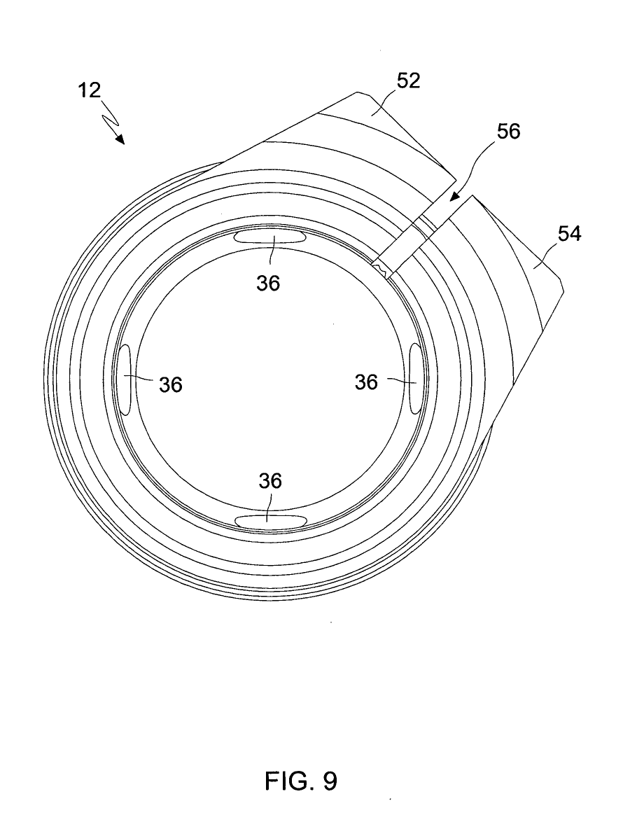

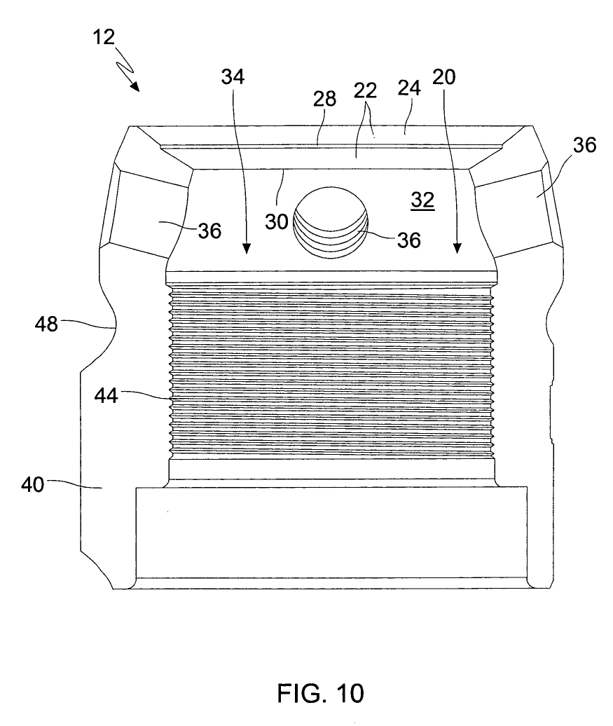

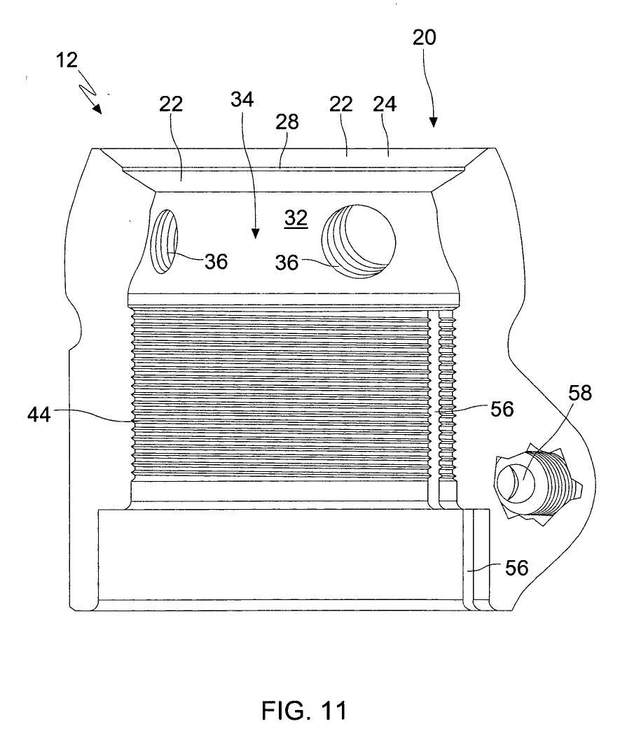

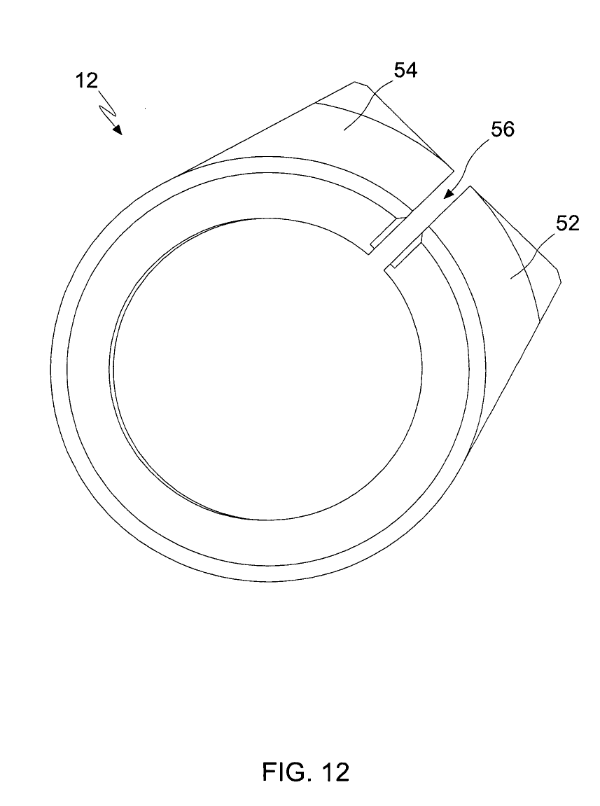

Referring to FIGS. 1-12, the exemplary proximal component 12 includes a proximal end pyramid receiver 16 having a generally cylindrically tapering exterior surface 18 defining a hollowed space 20 that extends axially through the entire component. The hollowed space 20 is at least partially defined by an interior circumferential wall 22 comprising a first ring 24 with an axially arcuate shape where the diameter of the ring decreases from proximal to distal (see FIG. 10). A distal end of this first arcuate ring 24 abuts a second arcuate ring 26, having a step change 28 in axial height therebetween.

This second arcuate ring 26 likewise includes an axially arcuate shape, where the diameter of the ring decreases from proximal to distal. The distal end of the second ring 26 includes a lip 30 that transitions into a tapering interior wall 32 that defines a frustoconical cavity 34, where the diameter of the interior wall 32 increases from proximal to distal. Four through holes 36, equidistantly spaced, extend through the pyramid receiver 16 and into the frustoconical cavity 34. Each hole 36 is slightly angled downward from, proximal to distal and is internally threaded to receive a set screw 38. By way of example, and not limitation, it is envisioned that the proximal end 16 receives a prosthetic pyramid (not shown), such as those available from Prosthetic Design of Clayton, Ohio (http://www.prostheticdesign.com/cms_pyra.html#tita), where the pyramid is retained within the frustoconical cavity by tightening the set screws 38 against the pyramid.

The proximal component 12 also includes a distal end 40 comprising an integral clamp 42. The clamp 42 includes a generally cylindrical interior surface 44 that is threaded, while the exterior surface 46 is partially cylindrical. A circumferential depression 48 on the exterior of the proximal component transitions between the proximal and distal ends 16, 40. A lock 50 is integrally formed into the clamp 42. The lock 50 includes mirror image members 52, 54 that are separated from one another by an axial groove 56 formed through the distal end 40. Each member 52, 54 includes a through hole 58, with one of the holes being internally threaded and axially aligned with respect to the opposing hole. In between the members 52, 54 is a removable key 60 that extends along the groove 56 and at least partially extends into an interior cylindrical opening 62 within the clamp 42. The key 60 includes a hole (not shown) therethrough that is adapted to be aligned with the through holes 58 of the members 52, 54 to receive a screw (not shown) to draw the members closed toward one another. In this fashion, the key 60 is pivotable about the screw so that a proximal portion 62 of the key may be actuated inward toward the interior of the clamp 42 and pivot outward a distal portion 64 of the key from the interior of the clamp. In other words, the key 60 is biased by a rubber cushion (not shown) positioned within the proximal end of the groove 56 to maintain the distal portion of the key projecting within the interior of the clamp 42.

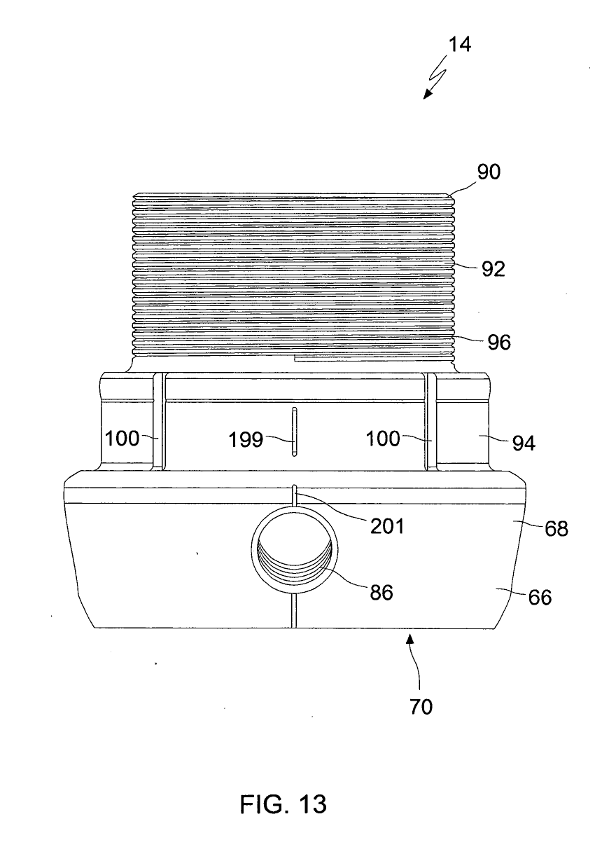

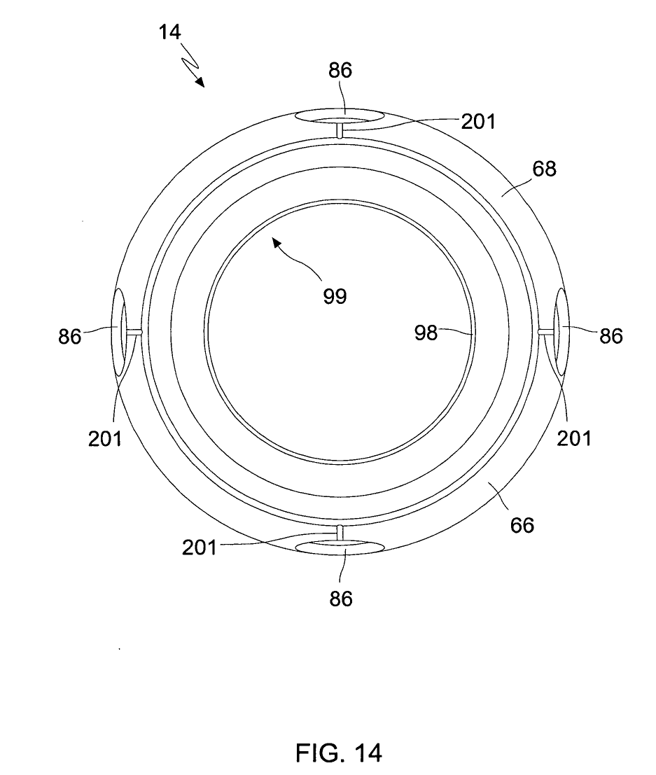

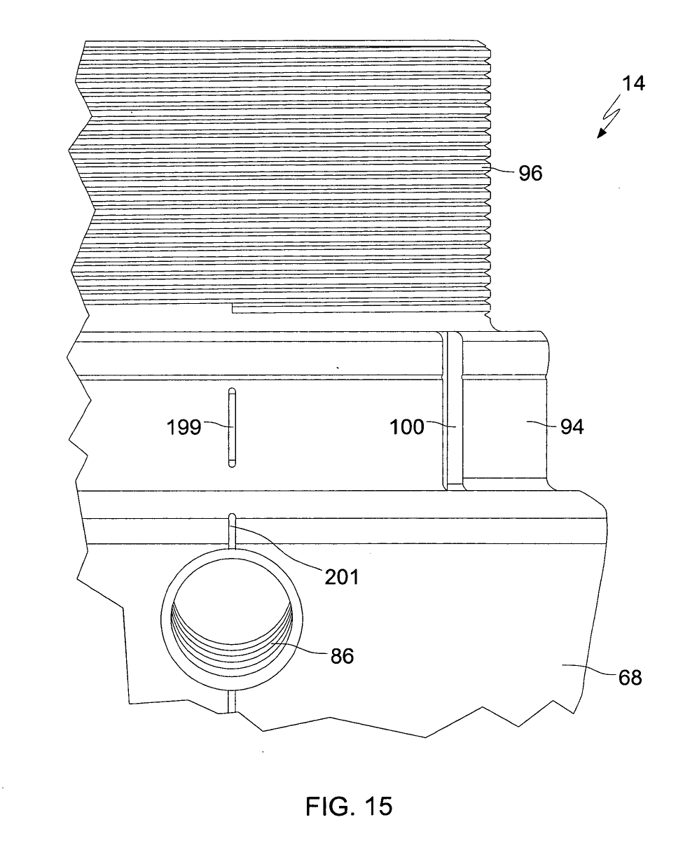

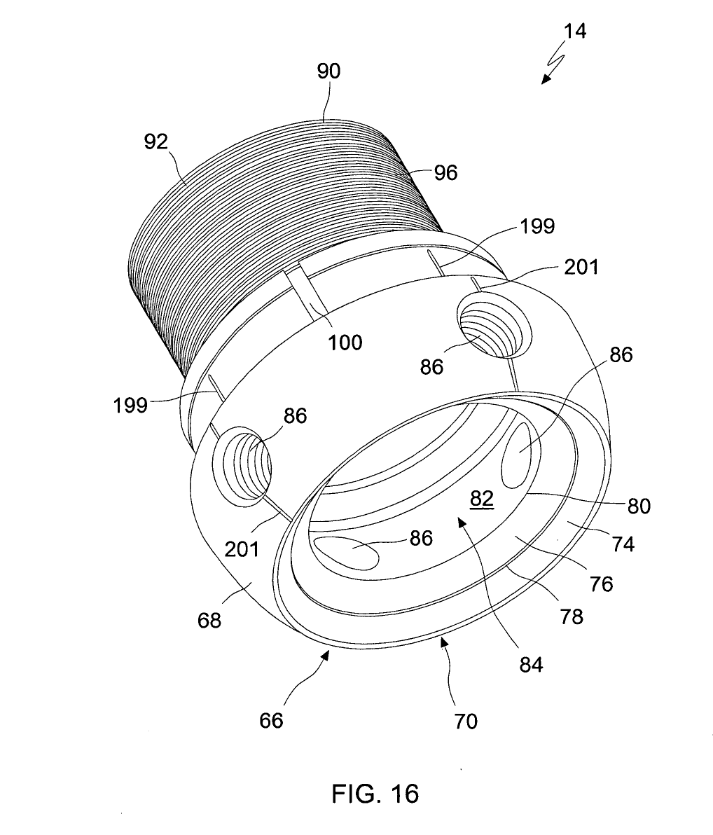

Referring to FIGS. 1-4 and 13-16, the distal component 14 includes a distal end pyramid receiver 66 having a generally cylindrically tapering exterior surface 68 defining an hollowed space 70 that extends axially through the entire component. The hollowed space 70 is at least partially defined by an interior circumferential wall 72 comprising a first ring 74 with an axially arcuate shape where the diameter of the ring, decreases from distal to proximal. A proximal end of this first arcuate ring 74 abuts a second arcuate ring 76, with a step change 78 in axial height therebetween toward the proximal direction. This second arcuate ring 76 likewise includes an axially arcuate shape where the diameter of the ring decreases from distal to proximal. The proximal end of the second ring 76 includes a lip 80 that transitions into a tapering interior wall 82 that defines a frustoconical cavity 84, with the diameter of the interior wall 82 increasing from distal to proximal. Four through holes 86, equidistantly spaced, extend through the distal end 66 and into the frustoconical cavity 84. Each hole 86 is angled upward from distal to proximal and internally threaded to receive a set screw (hot shown). By way of example, and not limitation, it is envisioned that the distal end 66 receives a prosthetic pyramid, such as those available from Prosthetic Design of Clayton, Ohio (http://www.prosmeticdesign.com/cmsjyra.html#tita), where the pyramid is retained within the frustoconical cavity 84 by tightening the set screws against the pyramid.

The distal component 14 also includes a proximal end 90 comprising a hollow cylinder 92 and ah interposing collar 94. The exterior surface 96 of the cylinder 92 is threaded, while the interior surface 98 is generally smooth. A distal endpoint of the threaded exterior 96 transitions into the collar 94, as denoted by a step change: increase in external diameter. In this exemplary embodiment, the collar 96 is simply an exterior feature, as the dimensions of the cylindrical cavity 99 within the proximal end 90 are generally uniform along the length of the cylinder 92 and collar 94 (i.e., axially along the distal component). A series of four notches 100, which are equidistantly spaced, extend axially along the exterior of the collar 94 and end approximate the distal end 66. The distal portion of the collar 94 transitions directly into the distal end 66 so that the entire distal component is integral. As will be discussed in more detail hereafter, at least one of the four notches 100 is aligned with the key 60 to fix the rotational position of the proximal component 12 with respect to the distal component 14 during a prosthetic fitting procedure.

This exemplary embodiment 10 allows for vertical and rotational adjustment by way of the interface between the proximal component 12 and the distal component 14. In an exemplary fitting procedure using the exemplary prosthetic adapter 10, a fitter may begin with the components 12, 14 separated from one another. Thereafter, the fitter would insert the exteriorly threaded portion 96 of the distal component 14 into the interiorly threaded portion of the proximal component 12 so that the threads engage one another, while at the same time depressing the proximal portion 62 of the key 60 to inhibit the distal portion 64 of the key from impeding rotation by becoming captured within one of the four notches 100. In sum, clockwise rotation of the distal component 14 (presuming the proximal component 12 is held stationary or turned counterclockwise) would draw the exteriorly threaded portion 96 of the distal component 14 further into the interiorly threaded portion 44 of the proximal component 12. As the exteriorly threaded portion 96 of the distal component 14 is further inserted into the interiorly threaded portion 44 of the proximal component, the vertical distance (along the axial direction of the adapter 10) between the proximal end 16 of the proximal component 12 and a distal end 66 of the distal component 14 is decreased. In this manner, if the fitter is desirous to decrease the distance between the respective ends 16, 66, the fitter would simply screw the distal component 14 further into the proximal component 12. Conversely, if the fitter is desirous to increase the distance between the respective ends 16, 66, the fitter would simply screw out (i.e., turn counter-clockwise the distal component 14 with respect to the proximal component) the distal component 14 from the proximal component 12. Once the proper vertical distance (or very close thereto) has been established, the distal component 14 is rotated with respect to the proximate component 12 in either the clockwise or counterclockwise direction so that the nearest of the four notches 100 is aligned and overlaps the groove 56. At this time, the proximal portion 62 of the key 60 is no longer depressed (i.e., it is released by the fitter), which allows the spring bias of the rubber cushion to push the proximal portion outward, away from the interior of the clamp 42. In so doing, the key 60 pivots around the screw of the lock 50 so that the distal portion 64 is directed toward the interior of the clamp 42 and into the aligned notch 100. When locked, the proximal component 12 is not rotatable with respect to the distal component 14 and, therefore, height adjustment (vertical distance between the proximal end 16 of the proximal component 12 and the distal end 66 of the distal component 14) between the components 12, 14 is also inhibited. In order to change the height and rotational orientation of the components 12, 14, the fitter would depress the proximal portion 62 of the key 60 and begin rotating the components with respect to one another in a clockwise or counterclockwise direction until the desired height is achieved. Thereafter, the proximal portion 62 of the key would be released so that the distal portion 64 would enter the corresponding notch 100 to secure the components 12, 14 in relative position.

After adjusting and locking the components in relation to one another, prior art methods may be utilized to secure the pyramids to the pyramid receivers 16, 66 on the ends of the components 12, 14. For purposes of explanation only, this procedure may include inserting the pyramid into the frustoconical cavity 34, 84 so that each face of the pyramid is perpendicular with respect to one of the holes 36, 86. In this manner, set screws 38 inserted into the threaded holes 36, 86 are tightened and ultimately abut the faces of thy pyramid, thereby compressing the pyramid on four sides the lock the pyramid in place with respect to the components 12, 14 and, thus, with respect to the adapter 10.

In the exemplary embodiment, adjustments are available in quarter-turns (i.e., when a notch 100 aligns with the groove 56). In this embodiment, each quarter-turn equates to approximately a 0.2 millimeter height adjustment ( 1/128 of ah inch). For example, it is within the scope of the invention to include more or fewer notches 100 depending upon the number of adjustment increments desired. Further, some or all of the notches 100 may be provided in the form of through holes, which may extend fully through the wall of the component.

In exemplary embodiments, various alignment or index marks may be provided at various points on the components 12, 14. For example, index marks 199, 201 are located on component 14 and index marks 203, 205 are on component 12. Exemplary embodiments include index marks 199, 201, 203, 205 substantially aligned with the holes 36, 86. Alignment of the index marks 199, 201, 203, 205 indicates that the key 60 is aligned with one of the notches 100. It is within the scope of the disclosure to include more or fewer index marks and to provide index marks at locations other than substantially aligned with the holes 36, 86.

Following from the above description and invention summaries, it should be apparent to those of ordinary skill in the art that, while the methods and apparatuses herein described constitute exemplary embodiments of the invention, the invention is not limited to this precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the invention may exist even though they may not have been explicitly discussed herein.

What is claimed is:

1. A prosthetic coupling comprising:
a proximal component including a first pyramid receiver and a first threaded portion; and
a distal component including a second pyramid receiver and a second threaded portion, the second threaded portion engaging the first threaded portion and allowing at least one of rotational and vertical adjustability between the first pyramid receiver and the second pyramid receiver.

2. The prosthetic coupling of claim 1, further comprising a lock operative to selectively inhibit at least one of rotational adjustability of the proximal component relative to the distal component and vertical adjustability between the proximal component and the distal component.

3. The prosthetic coupling of claim 2, wherein the lock includes a key mounted to one of the proximal component and the distal component and at least one notch on the other of the proximal component and the distal component; wherein the notch receives at least a portion of the key to inhibit at least one of rotational adjustability Of the proximal component relative to the distal component and vertical adjustability between the proximal component and the distal component.

4. The prosthetic coupling of claim 3, wherein the key is movably mounted to the one of the proximal component and the distal component; wherein the key is movable between an engaged position in which the key is at least partially received within the notch to inhibit least one of rotational adjustability of the proximal component relative to the distal component and vertical adjustability between the proximal component and the distal component, and a disengaged position allowing least one of rotational adjustability of the proximal component relative to the distal component and vertical adjustability between the proximal component and the distal component.

5. The prosthetic coupling of claim 4, further comprising a bias operative to urge the key towards the engaged position.

6. The prosthetic coupling of claim 5, wherein the key is pivotable between the engaged position and the disengaged position.

7. The prosthetic coupling of claim 6S wherein the at least one notch is located on a circumferential surface of the distal component at least partially between the second pyramid adapter and the second threaded portion.

8. The prosthetic coupling of claim 7, wherein the key is mounted to the proximal component and the key extends at least partially beyond the first threaded portion in a direction away from the first pyramid receiver.

9. (canceled)

10. The prosthetic coupling of claim 1, wherein the one of the first threaded portion and the second threaded portion includes an internally threaded, generally cylindrical cavity and the other of the first threaded portion and the second threaded portion includes an externally threaded projection.

11-16. (canceled)

17. The prosthetic coupling of claim 1, wherein the first pyramid adapter includes at least one threaded hole adapted to receive a set screw.

18. The prosthetic coupling of claim 1, wherein the proximal component includes a clamp, the clamp including a generally cylindrical axial opening including an interior surface that is at least partially threaded.

19. The prosthetic coupling of claim 18, wherein the generally cylindrical axial opening is partially threaded and partially smooth.

20. (canceled)

21. The prosthetic coupling of claim 20, wherein the clamp includes a through hole extending perpendicular to the groove.

22. The prosthetic coupling of claim 18, wherein the clamp is integrally formed with the first pyramid adapter.

23. A prosthetic coupling comprising:
a first component including a first pyramid adapter and an internal cavity having an opening;
a second component including a second pyramid adapter and a projection at least partially received within the internal cavity; and
a key mounted to one of the first component and the second component to selectively inhibit rotation between the first component and the second component.

24. The prosthetic coupling of claim 2-3, further comprising a notch on the other of the first component and the second component; wherein the notch is adapted to releasably receive at least a portion of the key.

25. The prosthetic coupling of claim 23, further comprising a through hole in the other of the first component and the second component; wherein the through hole is adapted to releasably receive at least a portion of the key.

26-30. (canceled)

31. The prosthetic coupling of claim 23, wherein the first component includes a first alignment mark and the second component includes a second alignment mark; and wherein alignment of the first alignment mark and the second alignment mark indicates that the key is aligned with the notch.

32. The prosthetic coupling of claim 31, wherein the first pyramid adapter includes at least one threaded hole and the first alignment mark is substantially aligned with the at least one threaded hole.

33-39. (canceled)

40. A prosthetic coupling adapter comprising:
a first component including
a first pyramid receiver/including a hollow axial opening and a plurality of generally radially oriented threaded holes extending to the hollow axial opening, and
a clamp mounted axially adjacent to the first pyramid receiver including
an axial cylindrical opening including an internally threaded portion and an internal smooth portion,
an axial groove extending from an exterior of the clamp to the axial cylindrical opening, and
a key pivotably mounted within the groove; and
a second component including
a second pyramid receiver including a hollow axial opening and a plurality of generally radially oriented threaded holes extending to the hollow axial opening, and
a projection mounted axially adjacent the second pyramid receiver including
an externally threaded portion engaging the internally threaded portion, and
an external smooth portion including at least one notch, the notch being selectively engageable with at least a portion of the key.

41. The prosthetic coupling adapter of claim 40, wherein the internal smooth portion has a diameter greater than a diameter of the internally threaded portion.

42. The prosthetic coupling adapter of claim 40, wherein the external smooth portion has a diameter greater than a diameter of the externally threaded portion.

43. The prosthetic coupling adapter of claim 40, wherein the first pyramid receiver includes a hollowed space at least partially defined by an interior circumferential wall, the interior circumferential wall including a first ring having an axially arcuate shape with an axially decreasing diameter towards a second arcuate ring, a step change in axial height interposing the first arcuate ring and the second arcuate ring.

44-49. (canceled)
